(19) United States
(12) Patent Application Publication
(10) Pub. No.: US 2003/0088883 A1
(43) Pub. Date: May 8, 2003

(54) Transgenic constructs for studying fibrotic diseases and uses thereof

(76) Inventors: Michael Breindl, San Diego, CA (US); David A. Brenner, Chapel Hill, NC (US)

Correspondence Address: MORRISON & FOERSTER LLP, 3811 VALLEY CENTRE DRIVE, SUITE 500, SAN DIEGO, CA, 92130-2332, US

(21) Appl. No.: 10/306,171

(22) Filed: Nov. 27, 2002

Related U.S. Application Data
(62) Division of application No. 09/745,902, filed on Dec. 22, 2000.

(51) Int. Cl. A01K 67/00; C07H 21/04; C12P 21/02; C12N 5/06

(52) U.S. Cl. 800/8; 435/69.1; 435/320.1; 435/325; 536/23.5; 530/356

(57) ABSTRACT

The present invention relates to the use of a reporter gene to study the tissue-specific expression of certain genes associated with fibrotic disease. More particularly, it relates to transgenic nucleic acid constructs having the reporter gene under operational control of the Colla1 promoter further comprising a regulatory element that regulates the expression of collagen. Such constructs and methods using these constructs are particularly useful in studying correct stage and tissue specific expression of collagen.

### STATEMENT OF RIGHTS TO INVENTIONS MADE UNDER FEDERALLY SPONSORED RESEARCH

This work was at least partially supported by NIH grants AR41909 (M.B.), AA10459 (R.A.R.), GB41804 (D.A.B.), and P50-AA11605 (R.A.R. and D.A.B.)

### TECHNICAL FIELD

The present invention relates to the use of a reporter gene, and in particular the gene encoding green fluorescent protein, to study the expression of certain genes that are associated with fibrotic diseases. More explicitly, the Colla1 gene is regulated by cis-regulatory elements, some of which are located in proximity to the start site of transcription whereas others have been identified at considerable distances. By exploiting the use of reporter genes in functional proximity to these regulatory elements, the present invention provides for particular reporter gene constructs containing the Colla1 promoter driving the green fluorescent protein (GFP) reporter gene. The study and design of these constructs in transfection experiments and animal models provides for novel ways of monitoring the progress and treatment of fibrotic diseases.

### BACKGROUND ART

The expression of eukaryotic genes is regulated by the concerted actions of proximal and distal cis-regulatory elements. While the proximal elements interact directly with the basic transcription machinery and trans-activating factors, distal elements are thought to exert their effects by establishing and maintaining a transcription-competent chromatin environment. A complete understanding of the stage- and tissue-specific regulation of a particular gene requires knowledge of the functions, modes of action of, and interaction between both types of elements.

One of the most widely studied eukaryotic gene families encodes the collagen “superfamily”, which contains at least 19 different collageneous proteins, and several more proteins which are known that contain collagen-like domains. Prockop, D. J. and Kivirikko, K. I. Annu. Rev. Biochem 64:403-434; 1995. Type I collagen, a fibrillar collagen, is the most abundant of the collagens and probably the most abundant protein in vertebrates. It has diverse biological functions; it provides tensile strength to connective tissues such as bone, skin, tendons, and ligaments; it forms supporting frameworks of connective tissue in all major internal organs such as liver, spleen, heart, and the vascular system; it promotes cell migration and differentiation during embryonic development; and it is the major substance produced during wound healing and in tissue repair processes. Thus, the Colla1 and Colla2 genes, which code for the α1 and α2 subunits of type I collagen, respectively, are expressed during embryonic development, in a variety of cell types, and under various physiological conditions, and their regulation is accordingly complex. (Bornstein, P. Matrix Biol. 15:3-10, 1996; Brenner, D. A., et al., J. Lab. Clin. Med. 124:755-760, 1994. Stack, J. D., et al., Am. J. Med. Genet. 45.140-151, 1993; Vuorio, E. and de Crombrugghe, B. Annu. Rev. Biochem. 59:837-872, 1990) Moreover, various human disorders, and in particular fibrotic diseases, are associated with under- or over-expression of type I collagen. An elucidation of the complex molecular mechanisms involved in the stage- and tissue-specific regulation of type I collagen expression is therefore not only essential for understanding normal mammalian development and morphogenes, but also the human disease process.

Numerous studies have addressed the molecular mechanisms involved in the regulation of type I collagen genes in various species, including human, rat, mouse, and chicken. However, despite considerable efforts, many details of type I collagen gene regulation remain elusive. It has generally been reported that the α1 and α2 promoters confer basic tissue-specific expression to reporter gene or minigene constructs in transfection experiments and transgenic animals (Rhodes, K., et al., Gene Expr. 6:35-44, 1996; Rippe, R. A., et al., Mol. Cell Biol. 9:2224-2227, 1989; Rossert, J. A., et al., J. Cell Biol. 129:1421-1432, 1995; Stack, J. D., et al., Am. J. Med. Genet. 45.140-151, 1993; Sokolov, B. P., et al., J. Biol. Chem. 270-9622-9629, 1995.) In addition, cis-regulatory elements have been identified that mediate the effect of various modulators of type I collagen expression (Inagaki, Y., et al., J. Biol. Chem. 296:14828-14834, 1994; Ritzenthaler, J. D., et al., Biochem J. 280:157-162, 1991, et al., J. Cell. Biochem, 58:380-392, 1995) or are required for high levels of expression in different collagen-producing cell types (Bedalov, A., et al., J. Bone Miner. Rec. 10:1443-1452, 1995; Rossert, J. A., et al., J. Cell Biol. 129:1421-1432, 1995; Rossert, J. A., et al., Proc. Natl. Acad. Sci. USA 93:1027-1031, 1996.)

Several lines of evidence indicate that, in addition to regulatory elements in the proximal promoter, more distal regulatory elements in both 5′- and 3′-flanking region may contribute to the correct stage- and tissue-specific expression of the type I collagen genes. A cluster of DNase I-hypersensitive sites in the distal 5′-flanking sequence of the murine Colla2 gene has been shown to have transcriptional enhancer activity in transgenic mice. (Bou-Gharios, G., et al., J. Cell Biol. 134: 1333-11344, 1996.) A chromatin structure analysis of the murine Colla1 gene has revealed the presence of several distal 5′ and 3′ DNase I-hypersensitive sites at positions very similar to the hypersensitive site in the homologous human COLIAL gene. (Barsh, G. S., et al., J. Biol. Chem 259:14906-14913, 1984; Salimi-Tari, P., et al., Gene 198:61-72, 1997.) Accordingly, this murine-minigene provides an excellent animal model for predicting the effects of certain conditions of the human gene. An E-box in the 3′-flanking region of the Colla1 gene binds transcription factors USF-1 and USF-2 and stimulates Colla1 gene transcription. (Rippe, R. A., et al., J. Biol. Chem. 272:1753-1760, 1997.) The fact that several of the distal 5′-hypersensitive sites represent in collagen-producing but not in nonproducing cells suggests that they function in Colla1 gene regulation. (Salimi-Tari, P., et al., Gene 198:61-72, 1997.) A number of such distal hypersensitive sites can be found -6 to-9 kb from the Colla1 promoter. These sites may contain an in vivo topoisomerase II cleavage site and a nuclear matrix attachment region. Accordingly, these sites may constitute the locus control region (LCR) of the Colla1 domain. LCRs are modular cis-regulatory elements, which are thought to be necessary for a position-independent, copy number-dependent, stage-and-tissue specific, and high-level expressions of a linked transgene. (Fraser, P., et al., Curr. Opin. Genet. Dev. 10:361-365, 1998; Kioussis, et al., Curr. Opin. Genet. Dev. 7:614-619, 1997.) However, a recent report that the much studied β-globin LCR is not necessary for an open chromatin structure or developmentally regulated transcription raises new questions about the exact nature and function of LCRs (Epner, E., et al., Mol. Cell 2:447-455, 1998.) In many cases, LCRs are composed of multiple DNase-hypersensitive sites with different functions (enhancers, insulators, chromatin opening elements, nuclear matrix attachment regions) that act in a synergistic manner.

Expression of a gene or transgene is said to be variegated when it is expressed only in a certain proportion of cells in a population of a given cell type, and the degree of variegation is position dependent and is thought to spread from regions of heterochromatin. In several instances cis-regulatory elements (enhancers, LCRs) have been shown to function by overcoming such heterochromatin-mediated position effect variegation (Festenstein, R., et al., Science 271:1123-1125, 1996; Sutherland, H. G. E., et al., Mol. Cell. Biol. 17:1607-1614, 1997; Walters, M. C., et al., Genet. Dev. 10.185-195, 1996.) The Colla1 upstream regulatory elements studied here, notably HS8.9, which may be part of a Colla1 LCR, were not able to prevent variegation. This confirms that they do not constitute an enhancer of the type that can overcome variegated expression or a fully functional LCR. (Sutherland, H. G. E., et al., Mol. Cell. Biol. 17:1607-1614, 1997; Walters, M. C., et al., Genes Dev. 10.185-195, 1996.) Moreover, variegation showed a tendency to increase with age of the animals, an observation also reported by others (Sutherland, H. G. E., et al., Mol. Cell. Biol. 17:1607-1614, 1997.) The phenomenon of variegated expression is of interest because it is possible that the regulation not only of transgenes, but also of endogenous tissue-specific genes, occurs through this mode (i.e., the number of cells in a given tissue modulating expression of a gene in response to physiological stimuli may vary rather than the level at which the gene is expressed in every cell). Similarly, it has been proposed that variegation may be involved in the acquisition and maintenance of particular cellular phenotypes (lineage commitment) during differentiation (Festenstein, R., et al., Science 271:1123-1125, 1996; Kioussis, D., et al., Curr. Opin. Genet. Dev. 7:614-619, 1997.) The use of the GFP gene as a reporter gene in transgenic mice offers several advantages over the use of other reporter genes such as the CAT, β-galactosidase, or luciferase genes. Because fluorescence analysis requires no fixation or staining, it is readily detectable using a fluorescent microscopic (or a hand-held lamp with appropriate filters) in live newborn adult animals. It can be microscopically analyzed in unfixed or fixed whole mount embryos, in whole organs or tissues such as tail, toes, uterus, skin, and bone, or (in highly expressing animals) kidney, heart, lung, and muscle. In fixed embryos and frozen organs or tissues, fluorescence remains detectable for many months. GFP can also be easily detected in histological analyses of thin sections of different organs or quantified very sensitively in tissue or cell extracts using a fluorometer. Finally, GFP expression can be observed in live transfected cells or primary cell cultures, such as dermal fibroblasts or marrow stromal cells, and the effect of modulators of (in this instance) Colla1 promoter activity can be monitored over long periods of time in such cultures.

Accordingly, the methodologies and transgenic mouse lines expressing the GFP gene driven by the Colla1 promoter described herein provide useful tools to study many aspects of the regulation of type 1 collagen and other extracellular matrix components.

### DISCLOSURE OF THE INVENTION

According to one aspect of the invention, a transgenic nucleic acid construct is provided that is useful for assessing collagen production associated with fibrotic disease, wherein said construct comprises nucleic acid sequences encoding: a Colla1 gene-specific regulatory element, wherein said regulatory element is associated with correct stage or tissue specific expression of collagen, and wherein said regulatory element is inserted into the construct at a far upstream DNAse I-hypersensitive site; a Colla1 gene promoter; and a reporter.

The transgenic nucleic acid construct is preferably insterted into the DNAse I-hypersensitive sites 3 to 9, more preferably into sites 4 to 8, and in representative embodiments, into sites.4.5 or 8.9.

Although reporters are well know in the art, a preferred reporter is green fluorescent protein.

In another aspect of the invention, the transgenic nucleic acid construct is constructed in a manner that causes expression of the reporter from the construct to be steroid hormone-dependent.

In yet another aspect of the invention, a method is provided for assessing collagen production associated with fibrotic disease comprising transfecting the construct hereinbefore described into a cell or non-human animal capable of expressing the reporter, and monitoring expression in said cell or non-human animal under varying conditions. Such fibrotic diseases include, inter alia, polycystic ovarian syndrome, uterine leiomyomas, cardiac fibrosis, and myocardial infarction-initiated fibrosis.

Alternative, a method is provided for assessing collagen production during different stages of embryotic development, comprising: constructing a transgenic nucleic acid construct, transfecting the construct into a non-human animal capable of expressing the reporter during different stages of embryotic development, and monitoring expression in said non-human animal during different stages of embryonic development.

Also provided is a method for assessing differential collagen production in different tissues, comprising: inserting the construct into a non-human animal capable of differentially expressing the reporter in different tissues, and monitoring expression of the reporter in different tissues in the non-human animal.

The invention also embodies a transgenic non-human animal comprising the construct hereinbefore described, as well as a cell and a vector.

Other aspects of the invention are described throughout the invention, and this section should not be interpreted as a complete description of what applicants consider to be their invention.

### BRIEF DESCRIPTION OF THE DRAWINGS

FIG. 1 depicts several different reporter gene constructs.

FIG. 2 depicts the effects of the Colla1 promoter activity in stable transfections.

FIG. 3 depicts the ability of various constructs to express GFP.

FIG. 4 depicts the relative GFP expression in uteri and tails of trasngenic mice.

FIG. 5 depicts the correlation between relative GFP expression in the tails of transgenic mice and percentage of GFP-expressing cells in primary dermal fibroblast cultures.

### BEST MODE FOR CARRYING OUT THE INVENTION

The present invention relates to the design and use of transgenic gene constructs and animal models for studying the regulation of Colla1 gene expression. As such, the present invention provides for tools and methods for evaluating the diagnosis and/or treatment of collagen expression-associated fibrotic diseases.

Many different models currently exist for the study of differential expression of proteins in various cell types and the regulation of such expression by genetic control elements. For example, in Mol. Endocrinol., 12(5): 622-633, 1998, Brinkeier et al., disclose the regulation of the glycoprotein hormone alpha-subunit gene in pituitary gonadotropes and thyrotropes, and its control by different expression enhancement elements.

One of the easiest ways to study the function of regulatory elements and the production of a particular protein under the control of such regulatory elements is to use gene constructs which contain both the structural gene promoter, the regulatory element being investigated, and a reporter gene, the expression of which under the control of the promoter can be easily monitored.

Thus, according to the present invention, the steps involved in developing a model system for studying fibrotic disease are:

Idenitification of the genetic sequence encoding a regulatory element to be studied and its location proximal to the structural gene, which in a preferred embodiment is a far upstream cis-regulatory element, such as the DNase I-hypersensitive site which regulates the expression of the murine alpha-I (I) collagen (Colla1) gene

Cloning of the genetic sequence encoding the regulatory element into a gene construct, along with a reporter gene under the control the promoter for the structural gene

Designing experimental models by performing transfection experiments and/or constructing transgenic animal models

Testing the function of the regulatory elements in the experimental models under various experimental conditions.

What follows is a discussion of each of these steps as they pertain to the present invention. Although much of the following discussion relates to a preferred embodiment of the present invention, it should be understood by those of skill in the art that many of the techniques and protocols presented herein are easily adapted and optimized for a variety of different experimental conditions. These include the choice of vector, the precise location of insertion of genetic elements, the conditions of expression, etc.

Regulatory Element Identification

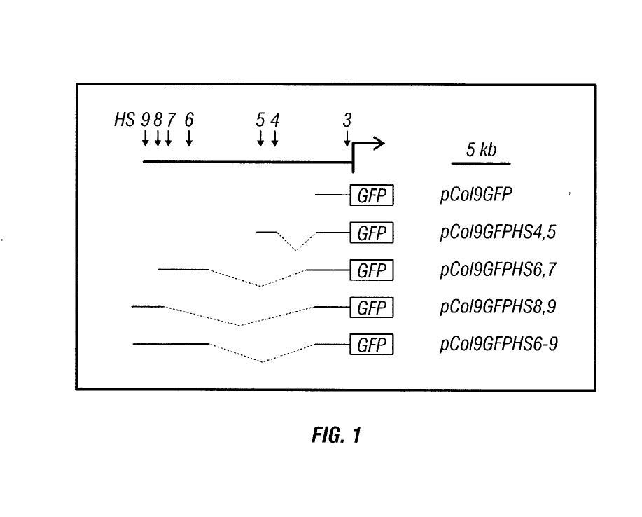

Regulatory elements that control the expression of structural genes can easily be identified and cloned using routine cloning techniques. As described below in Example 1, a series of distal DNase-hypersensitive sites in the 5′-flanking region of the murine Colla1 gene have previously been described and are depicted in FIG. 1. (Salimi-Tari, P., et al., Gene 198:61-72, 1997.) The fact that most of these sites were found in collagen-producing 3T3 fibroblasts and osteoblasts but not in nonproducing WEHI 3B myclomonocytic leukemia cells strongly suggests that they function in the regulation of Colla1 gene expression.

Preparation of Gene Constructs

To analyze their possible functions, as described elsewhere herein, a series of reporter gene constructs were prepared containing the Colla1promoter from −1622 to +111, the luciferase reporter gene, and the individual DNase-hypersensitive sites 4, 5, 6, 7, and 8 in both orientations as well as DNase-hypersensitive sites 4+5 and 6+7 in both orientations. The reporter genes were transiently transfected into NIH 3T3 fibroblasts and luciferase activity was determined after 48 h, normalized to transfection efficiency using a cotransfected β-gal reporter plasmid as described and compared to luciferase activity of a reporter gene containing the Colla1 promoter only. (Rhodes, K., et al., Mol., Cell Biol. 14:5950-5960, 1994.) None of the DNase-hypersensitive sites tested in this series of experiments had a significant effect on Colla1 promoter activity; HS 6 showed in approximately two-fold reduction and HS 8 an approximately twofold stimulation of reporter gene expression.

Designing an Experimental Model

Some regulatory elements exert their effect through an alteration of chromatin structure and can therefore only be detected in stable transfection after integration into the host cell DNA. Other elements regulate gene expression in very specialized tissues or cell types and cannot be detected in transfection experiments at all, but require introduction into transgenic animals. Accordingly, when studying the effects of various regulatory elements, it is preferred to construct transgenic animal models, in addition to cellular transfections. When such transgenic animal models were constructed based on the transfection experiments described above, new regulatory elements important for expression in some tissues in transgenic animals were discovered in the Colla1 promoter between −900 and −3200 (Rossert, J. A., et al., J. Cell Biol. 129:1421-1432, 1995.) Based on this, a new series of reporter gene constructs were prepared that included these additional promoter sequences for use in stable transfections and transgenic mice. These elements have no effect in Colla1 promoter activity in skin fibroblasts and therefore should not have affected the results of the transient transfection experiments, which were performed in fibroblasts. Although many different reporter genes are known in the art and could easily be adapted for use in the present invention, the green fluorescent protein (GFP) gene is preferred, because of the ease in which it can be optimized for expression in mammalian cells.

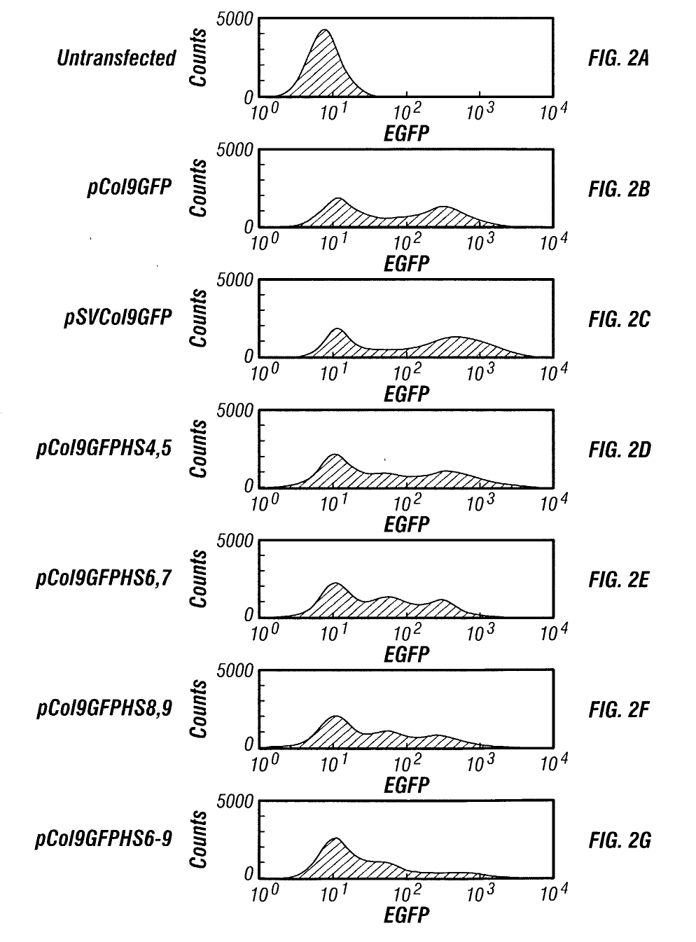

Accordingly, the reporter gene construct, pCol9GFP, was prepared which contains the Colla1 promoter from −3122 to +111 and the GFP reporter gene and several derivatives containing the various upstream DNase-hypersensitive sites as shown in FIG. 1, or the SV40 enhancer. This gene construct was used to perform transient and stable transfection experiments as described in the Experimental section. When the constructs were stably transfected into NIH 3T3 cells and GFP expression analyzed by FACS analysis we found that the SV40 enhancer had a slightly stimulating effect on Colla1 promoter activity whereas the various upstream DNase-hypersensitive sites showed a slightly inhibitory effect on the level of GFP expression and little or no effect on the number of GFP-expressing cells (FIG. 2).

As described in Example 3, three of the reporter gene constructs were chosen for initial introduction into transgenic mice: 1) the promoter-only construct pCol9GFP as a control; 2) pCol9GFPHS-4.5 because of preliminary evidence that DNase-hypersensitive sites 4 and/or 5 may contain a transcriptional enhancer; and 3) pCol9GFP-HS8,9 because HS 8 contains a topoisomerase II cleavage site and HS 9 a nuclear matrix attachment region (MAR),and these sites therefore resemble chromatin domain border elements or locus control regions (LCRs; a manuscript describing these findings is in preparation). Multiple transgenic mouse lines were established for each of the constructs and GFP expression was analyzed during embryonic development and in adult animals. Transgene-positive founder animals were identified by PCR amplification of GFP sequences in tail DNA, and transgene-expressing founders and off-spring were identified by inspection of GFP expression in tail clippings, toe clippings, or whole live animals under a fluorescent stereomicroscope (in some very highly expressing animals GFP was detectable as a greenish stain with the naked eye without fluorescent light). Expression in the tail was higher than in any other tissue in all founders with all three constructs and in all offspring analyzed (except in the uteri of some animals containing pCol9GFPHS-4.5; see below). Moreover, an analysis of a large number of PCR-positive but tail expression-negative animals revealed no indication of aberrant transgene expression in any of the lines (i.e., no animal lacking microscopically detectable fluorescence in the tail expressed the transgencs in other tissues when assayed by the very sensitive fluorimetric analysis of tissue extracts). Thus, fluorescence in the tail was a very reliable indicator of transgene expression for all three constructs.

Testing Gene Expression in the Experimental Model

Part I: Copy Numbers in Expressing Cell Lines

Transgene copy numbers in expressing lines were determined by Southern blot Analysis of liver DNA. Different lines harbored between 1 and >100 transgene copies (Table 1), and in most lines the Southern blot hybridization patterns were consistent with multiple transgene copies arranged in a head-to-tail fashion at a single integration site.

TABLE 1
COPY NUMBER AND GFP EXPRESSION IN TRANSGENIC
MOUSE LINES HARBORING DIFFERENT REPORTER
CONSTRUCTS

| Mouse Line | rel GFP* | % Cells+ | Copy No | GFP Copy |
|---|---|---|---|---|
| pCo19GFP | | | | |
| 2111 | 5.1 | 12 | 4 | 1.3 |
| 2116 | 10.7 | 17 | 2 | 5.3 |
| 2118 | 28.6 | 14 | 10 | 2.9 |
| 2125 | 11.5 | 35 | 2 | 5.8 |
| 2131 | 9.0 | 15 | 2 | 4.5 |
| 2132 | 4.2 | 3 | 6 | 7.0 |
| pCo19GFP-HS4.5 | | | | |
| 2018 | 83.9 | 73 | 7 | 12.0 |
| 2023 | 11.7 | 6 | ˜35 | 0.3 |
| 2024 | 8.2 | 60 | 1 | 8.2 |
| 2025 | 18.3 | 35 | 6 | 3.1 |
| 2027 | 17.7 | 37 | <100 | 0.2 |
| 2031 | 3.4 | 15 | 20 | 0.2 |
| 2033 | 100 | 90 | 50 | 2.0 |
| 2036 | 93.3 | 77 | 50 | 1.9 |
| 2048 | 31.4 | 37 | 10 | 3.1 |
| 2052 | 3.0 | 0 | 4 | 0.9 |
| pCo19GFP-HS8.9 | | | | |
| 2063 | 14.0 | 52 | 1 | 14.0 |
| 2606 | 11.0 | 30 | 14 | 0.8 |
| 2608 | 6.7 | 0 | 2 | 3.4 |
| 2610 | 40.6 | 31 | 10 | 4.1 |
| 2617 | 50.0 | 50 | 5 | 10.0 |
| 2623 | 23.3 | 43 | 3 | 7.8 |
| 2624 | 0.9 | 0 | 6 | 0.2 |

*GFP expression was measured in tail extracts from each individual founder animal and normalized to expression in the highest expressor 2033 (100%).
+Percent of GFP-expressing cells in primary dermal fibroblast cultures from each of the founders.

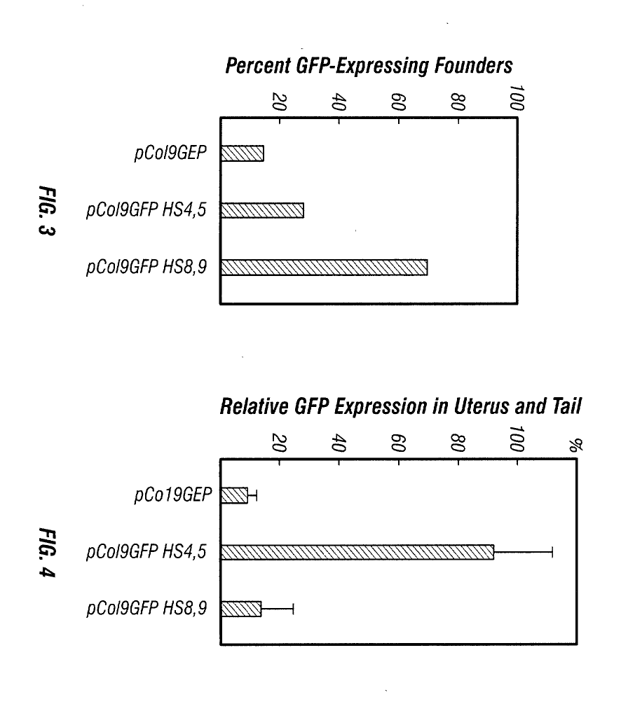

As shown in Table 1, the GFP expression in the tail of all founders at similar ages was compared and it was found that expression in the different lines varied ˜100 fold. The relative level of expression in a given line was stable (i.e., was very similar in several generations of off-spring). In addition, the level of GFP expression showed a good correlation to the proportion of cells in primary dermal fibroblasts that express the transgene. There was no classical enhancement of expression by HS4,5 or HS8,9 and GFP expression per copy number varied greatly in the different lines with each of the constructs (i.e., there was no copy number dependence of expression). There was, however, a significant increase in the position-independent transgene expression in mouse lines harboring the construct pCol9GFP-HS8,9 (FIG. 3). While construct pCol9GFP was expressed in 16.7% and pCol9GFP-HS4,5 in 31.3% of the PCR-positive founders, construct pCol9GFP-HS8,9 was expressed in 66.7% of the PCR-positive founders.

Part II: Basal Stage- and Tissue-Specific Expression of the GFP Reporter Gene

A transgenic embryo and a negative littermate at day 18 of embryonic development were investigated for expression of GFP. High levels of expression were observed in tendon (tail), bone (calvarium, ossification centers in the digits), and skin. Expression patterns at this level of analysis were undistinguishable in embryos containing the different constructs. Transgene expression at different stages of development was also evaluated. Expression was detectable throughout embryos at days 12 and 14 of development. At day 16 the highest expression was seen in bone and at days 17 and 18 in tail, skin, and bone.

Expression of the three reporter constructs was also analyzed in adult mice, and the results are given below in Table 2. Highest expression of all constructs was seen in the tail, followed by skin and bone; all major internal organs expressed at much lower, although clearly detectable, levels. Again, there were no significant differences in the relative levels of expression of the three different constructs in different tissues except for the significant higher levels of GFP in the uteri of mice containing the construct pCol9GFPHS-4,5.

TABLE II
OFP EXPRESSION IN TISSUE EXTRACTS OF ADULT TRANS-
GENIC MICE, HARBORING DIFFERENT REPORTER
CONSTRUCTS

| Construct | pCo19GFP | pCo19GFP-HS4,5 | pCo19GFP-HS8,9 |
|---|---|---|---|
| Tail | 100 | 100 | 100 |
| Bone | 16.9 (12.8) | 23.5 (7.7) | 21.7 (7.8) |
| Skin | 10.5 (7.6) | 41.8 (15.2) | 33.8 (14.1) |
| Muscle | 0.7 (0.3) | 1.7 (0.8) | 1.3 (0.4) |
| Heart | 0.3 (0.2) | 0.7 (0.5) | 0.8 (1.4) |
| Spleen | 0.3 (0.1) | 0.7 (0.3) | 0.2 (0.2) |
| Kidney | 0.3 (0.6) | 1.1 (0.9) | 1.8 (1.6) |
| Liver | 0.3 (0.2) | 0.2 (0.1) | 0.1 (0.0) |
| Lung | 0.1 (0.1) | 1.3 (0.8) | 0.2 (0.2) |
| Brain | 0.3 (0.0) | 0.6 (0.4) | 0.4 (0.2) |
| Thymus | n.d. | n.d. | 0.3 (0.1 |
| Uterus | 9.6 (5.1) | 91.0 (41.3) | 13.0 (19.8) |

GFP expression was measured in tissue extracts from one or several offspring of each of the founder animals and normalized to expression in the tail (100%). For each tissue the mean relative expression is shown with the SD in parentheses. n.d. = not determined.

Part III: Upstream DNase-Hypersensitive Sites 4,5 Specifically Enhance Colla1 Promoter Activity in the Uteri of Transgenic Mice

Female mice containing the construct pCol9GFP-HS4,5 showed a significant enhancement of GFP expression in the uterus relative to GFP expression in the tail (91.%) compared to mice containing the promoter-only construct pCol9GFP (9.6%; table 2, FIG. 6). The level of expression in uterus in these mice exceeded that in skin and bone of the same mice and was seen in offspring of all the different founders (i.e., was no position dependent).

Moreover, no enhancement of uterus-specific expression was seen in mice containing construct pCol9GFP-HS-8,9 (13.0%, Table 1, FIG. 4), and the enhancement was thus specific for pCol9GFP-HS4,5. These results suggest that DNase-hypersensitive sites 4 and/or 5 contain a novel transcriptional enhancer that may be involved in the regulation of type I collagen expression in tissue remodeling in the uterus during the estrous cycle.

To determine which uterine cells expressed the GFP reporter gene, thin sections from the uteri of sexually mature female transgenic mice were analyzed by fluorescent microscopy. High levels of GFP were detectable in endometrial stromal cells and the myometrium, but not in luminal epithelial cells or uterine glands. Similar expression patterns were observed in the uteri of transgenic mice containing the other constructs, although expression was less intense.

It is not readily apparent from these results at what stage of the estrous cycle the mice were at the time of analysis and how GFP expression might change throughout the estrous cycle, however, it will be apparent to one of skill in the art that this model is useful for analyzing the effect of female sex steroid hormones on the expression of the different reporter constructs.

Although not wishing to be bound by any particular scientific theory, the fact that the construct pCol9GFP-HS4,5 was expressed approximately 10-fold higher levels in the uteri of transgenic mice than the other constructs (Table 2, FIG. 6) suggests that hypersensitive sites 4 and/or 5 contain a tissue-specific enhancer that may be involved in the steriod hormone-dependent stromal cell proliferation and extracellular collagen deposition during the estrous cycle in the uterus and possibly in other organs or tissues. During the proestrous phase of the estrous cycle the ovarian hormones estrogen and progesterone stimulate growth and proliferation of the endometrium, which decreases in size in the postestrous phase and is sloughed unless implantation occurs. Thus, there is a constant remodeling of connective tissue, blood vessels, glands, and other parts of the uterus throughout the estrous cycle. The transgenic mice analyzed in this study presumably were in various stages of the estrous cycle and were synthesizing connective tissue at widely varying rates at the times of analysis. That would explain the large variation in GFP expression observed in the uteri (Table 2). Estrogen is known to affect type I collagen synthesis in the uterus (Dyer, R. F., et al., 1980; Mariotti, A., et al., Eur. J. Oral Sci. 106:1022-027, 1988); osteoblasts and osteosarcoma cells (Mahonen, A.; et al., J. Cell. Biochem, 68-151-163, 1998; Qu, Q., et al., Bone 22:201-209, 1998); and other tissues (Chang, W. Y., et al., Endicrinology 140:405-415, 1999.) The effect of 178-estradiol on collagen and noncollagenous protein synthesis in the uterus and some periodontal tissues. (Fischer, G. M., et al., Endocrinology 93:1216-1224, 1973.) Estrogen also induces expression of the progesterone receptor, and progesterone plays a role in the proliferation and differentiation of the endometrial stroma.

It is not known whether this effect of estrogen or progesterone on collagen synthesis is direct or requires other mediators. The DNA sequences requirements for estrogen receptor binding to estrogen response elements have been very well defined (Driscoll, M.D., et al., J. Biol. Chem. 273:29321-29330, 1998), while progesterone response elements are less well characterized (Graham, J. D. and Clarke, C. L. in Endocr. Rev. 18:502-519, 1997.)

Part IV: Different Levels of Transgene Expression Result from Different Degree of Variegation

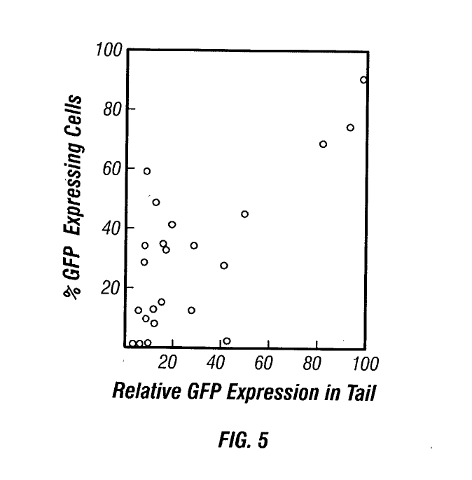

Different levels of transgene expression in different cell or mouse lines can result either from different levels of expression in every cell of a given population or from different proportions of cells within a given population expressing the transgene (variegated expression). Several cis-regulatory elements have been described that prevent variegated expression (Festenstein, R., et al., Science 271:1123-1125, 1996; Sutherland, H. G. E., et al., Mol. Cell. Biol. 17:1607-1614, 1997; Walters, M. C., et al., Genes Dev. 10.185-195, 1996.) In order to find out what determines the different expression levels of the constructs in the different lines (which was not related to transgene copy number, Table 1) and to examine whether the DNase-hypersensitive sites analyzed here had any effect on variegated expression of the linked Colla1 promoter, primary fibroblast cultures were prepared from each of the founders and the proportions of GFP-expressing cells were quantified for each line. The results are shown in Table 1 and FIG. 5. With some exceptions, there was a good correlation between the relative fluorescence in tail extracts and the number of GFP-positive cells, indicating that the level of transgene expression in most of the lines is determined primarily by variegation. Moreover, this correlation was seen with all three constructs used (i.e., none of the DNase-hypersensitive sites analyzed affected variegated expression of the linked transgene).

Use of Experimental Models to Study Particular Disease States

Polycystic ovarian syndrome: Polycystic ovarian syndrome (PCOS) is characterized by abnormal menstrual bleeding, hirsutism, obesity and infertility. Ovarian cysts are surrounded by a thickened thecal cell capsule which consists mainly of type I collagen and other connective tissue components, and it is thought that the increased amount of connective tissue in the capsule is one of the factors for anovulation. New nonsurgical treatment strategies for PCOS and leiomyomas have recently been proposed which target growth and transcription factors involved in stimulating connective tissue deposition in these disorders. In mice, neonatal estradiol injections leads to cystic ovaries similar to those in patients with PCOS. The collagen-GFP transgenic mice are used to determine which regulatory elements are involved in collagen upregulation in PCOS. Neonatal mice are injected on days 1 to 4 with 10 μl sesame oil containing 20 μg estradiol. Mice are sacrificed at various time points (4-8 weeks) and symptoms of PCOS are analyzed by regular and fluorescent microscopy of thin sections of the ovaries.

Uterine leiomyomas (fibroids): Uterine fibroids are smooth muscle cell tumors which are found in 20-25% of premenopausal women. They can be associated with infertility and can induce abortion. They are the most frequent reason for hysterectomy and pose a significant public health problem. Like other fibrotic diseases uterine fibroids are characterized by abundant amounts of extracellular matrix, notably type I collagen. As mentioned above, new nonsurgical treatment strategies for uterine fibroids and PCOS have recently been proposed which target growth and transcription factors involved in stimulating connective tissue deposition in these disorders. The Collagen-GFP transgenic mice are used to determine which regulatory elements are involved in collagen upregulation in uterine fibroids. In rodents, uterine fibroids are induced by estrogen treatment of ovariectomized females. Mice receive implants of estrogen-releasing pellets 7-10 days after ovariectomy. Mice are sacrificed at various time points (6-12 weeks) and analyzed by regular and fluorescent microscopy for the presence of uterine fibroids and excess collagen deposition.

Cardiac fibrosis: Hypertensive cardiovascular disease is associated with cardiac fibrosis which is characterized by changes in cardiac morphology, interstitial and perivascular collagen deposition, inflammatory cell infiltration and apoptosis. The collagen-GFP transgenic mice are used in the classical rodent model of mineralocorticoid-salt experimental cardiac fibrosis. In this approach, uninephrectomized mice are given high salt (0.9% NaCl/0.3% KC1) drinking water and implants of pellets releasing constant amounts of deoxycorticosterone acetate (DOCA) in the s.c. space of the intrascapular area. Animals are sacrificed at various time points after treatment (4-8 weeks) and symptoms of cardiac fibrosis are analyzed by fluorescent microscopy of thin sections of the hearts and fluorometry of tissue extracts.

Myocardial infarction: Myocardial infarction (MI) initiates a complex reparative process that results in a dense, highly organized collagenous scar, but the factors involved in collagen upregulation are poorly defined. The collagen-GFP transgenic mice are used to experimentally induce MI by coronary occlusion. Left thoroctomy is performed and ligature placed around the main coronary artery. Mice are sacrificed at various time points (24 hrs to 1 week) and analyzed for GFP expression by fluorescent microscopy and fluorometry Numerous modifications may be made to the foregoing inventions without departing from the basic teachings thereof. Unless defined otherwise, all technical and scientific terms used herein have the same meaning as is commonly understood by one of ordinary skill in the art to which this invention belongs. Although the present invention has been described in substantial detail with reference to one or more specific embodiments, those of skill in the art will recognize that changes may be made to the embodiments specifically disclosed in this application, yet these modifications and improvements are within the scope and spirit of the invention as set forth in the claims which follow. All publications, patents, and patent applications cited in this specification are incorporated herein by reference as if each such publication, patent, or patent application was specifically and individually indicated to be incorporated herein by reference.

### EXAMPLES

### Example 1

### Reporter Gene Constructs

The construction of the reporter genes used in this study required multiple cloning steps, the details of which have been described (10). Briefly, two different sets of reporter genes were used. The first set contained the Colla1 promoter from −1622 (PstI) to +111 (XbaI), the luciferase reporter gene, and various combinations of upstream DNase-hypersensitive sites. These constructs were made by replacing the SV40 promoter in the plasmid pGL2 (Promega) with the Colla1 promoter. A unique NotI site was then inserted upstream of the promoter into which NotI cassettes containing the upstream hypersensitive sites were cloned. The second set of reporter genes was constructed by cloning the Colla1 promoter from −3122 (KpnI) to +111 (XhaI) and the EGFP reporter gene from the plasmid pEGFP-1 (Clontech) into pPCR-Script (Stratagene). This plasmid (pCol9GFP) was then further modified to allow insertion of the NotI cassettes containing the upstream hypersensitive sites into a unique NotI site, and the subsequent removal of all vector sequences using two newly introduced SrfI sites to prepare DNA constructs for the generation of transgenic mice. Various DNA fragments containing the previously described Colla1 upstream hypersensitive sites (31) were cloned into pBluescript (Stratagene). Second NotI sites were then inserted into the plasmids so that the inserts were flanked by two NotI sites and could be recovered and cloned into the reporter genes as NotI cassettes. Orientation of all inserts and identity of the constructs were verified by multiple restriction enzyme digests and DNA sequence analysis when necessary. The plasmid pSVCol9GFP was made by cloning the Colla1 promoter from −3122 (KpnI) to +111 (XhaI) into the plasmid pEGFP-1 (Clontech), which contains the SV40 enhancer. A schematic representation of the GFP reporter constructs is shown in FIG. 1. The luciferase reporter constructs were used only in transient transfection assays, the results of which are not shown, and they are therefore not included in FIG. 1.

### Example 2

### Transient and Stable Transfections and Reporter Gene Assays

Transient and stable transfections and luciferase reporter gene assays were performed as previously described (24,25). GFP reporter gene expression in transfected cells was analyzed by visual inspection using an olympus inverted fluorescent microscope, by determining fluorescence in cell extracts using a TD700 fluorometer, or by fluorescence-activated cell sorting using a FACScan (Becton-Dickinson). Cell extracts for fluorescence measurements were prepared as described below for tissue extracts. For fluorescence-activated cell sorting the cells were trypsinized and resuspended in PBS.

### Example 3

### Generation of Transgenic Mice

Transgenic mice were generated using standard techniquies. DNA sequences containing the various reporter gene constructs were isolated from vector sequences by gel purification and injected into single-cell mouse embryos. The embryos were then reimplanted into pseudopregnant C3H×C57B1 foster mothers. At approximately 3 weeks of age tail clippings were obtained from the ups and analyzed for GFP expression using a fluorescent microscope. Subsequently, DNA was extracted from the tail clippings and presence of the transgene was determined using PCR and GFP-specific primers. Transgene copy numbers were determined by Southern blot analysis of liver DNA using standard procedures.

### Example 4

### GFP Expression in Whole Mount Embryos, Tissues and Primary Fibroblast Cultures from Transgenic Mice

Embryos were collected at different stages of development, day 1 being the day after the vaginal plug. The embryos were fixed in PBS containing 4% paraformaldehyde and 0.2% Tween 20 and stored at 4° C. Fluorescence was stable under these conditions for many months. For GFP analysis in tissue thin sections of the tissues were fixed for 24 h at 4° C. in PBS containing 4% paraformaldehyde. Blocks of tissues were mounted in O.C.T. compound (Lab-Tek) on specimen holders, frozen in liquid nitrogen, and cut into thin sections using a cryotome. Sections were mounted on Superfrost/Plus microscope slides (Fisher), stained with DAPI for 30 s, covered with FluorSave reagent (Calbiochem), and analyzed under a fluorescent microscope using DAPI and GFP-specific filter sets. To analyze transgene expression in tissues animals were sacrificed at between 4 and 20 weeks of age and tissues harvested and homogenized with a polytron homegenizer in 1 ml PBS containing 0.5% Triton X100, 10 μg/ml leupeptin, 20 μg/ml aprotinin, and 0.1 mM PMSF. Homogenates were microcentrifuged for 20 min and the supernatants removed to determine protein concentrations using a BCA protein assay reagent (Pierce) and fluorescence using a TD700 fluorometer. To prepare primary fibroblast cultures small pieces of skin were obtained from the ears of mice, sterilized in 70% ethanol, washed in PBS, and minced into small pieces with a scalpel. The pieces were then incubated in 200 U/ml collagenase at 37° C. for 24 h and subsequently in trypsin at 37° C. for 30 min, and cells were collected by centrifugation and grown in minimal essential medium containing 10% fetal calf serum.

We claim:

1. A transgenic nucleic acid construct useful for assessing collagen production associated with fibrotic disease, wherein said construct comprises nucleic acid sequences encoding:
a) a Colla1 gene-specific regulatory element, wherein said regulatory element is associated with correct stage or tissue specific expression of collagen, and wherein said regulatory element is inserted into the construct at a far upstream DNAse I-hypersensitive site;
b) a Colla1 gene promoter; and
c) a reporter.

2. The transgenic nucleic acid construct according to claim 1, wherein the site of insertion is the DNAse I-hypersensitive site 3 to 9.

3. The transgenic nucleic acid construct according to claim 2, wherein the site of insertion is the DNAse I-hypersensitive site 4 to 8.

4. The transgenic nucleic acid construct according to claim 3, wherein the site of insertion is the DNAse I-hypersensitive site 4.5.

5. The transgenic nucleic acid construct according to claim 2, wherein the site of insertion is the DNAse I-hypersensitive site 8.9.

6. The transgenic nucleic acid construct according to claim 1, wherein the reporter is green fluorescent protein.

7. The transgenic nucleic acid construct according to claim 1, wherein expression of the reporter from the construct is steroid hormone-dependent.

8. A transgenic nucleic acid construct useful for assessing collagen production associated with fibrotic disease, wherein said construct comprises nucleic acid sequences encoding:
a) a Colla1 gene-specific regulatory element, wherein said regulatory element is inserted into the construct at a far upstream DNAse I-hypersensitive site, and wherein the regulatory element is steroid hormone-dependent;
b) a Colla1 gene promoter; and
c) a reporter.

9. A method for assessing collagen production associated with fibrotic disease, comprising:
a) constructing a transgenic nucleic acid construct, wherein said construct comprises nucleic acid sequences encoding:
i) a Colla1 gene-specific regulatory element, wherein said regulatory element is inserted into the construct at a far upstream DNAse I-hypersensitive site;
ii) a Colla1 gene promoter; and
iii) a reporter; and
b) transfecting the construct into a cell or non-human animal capable of expressing the reporter; and
c) monitoring expression in said cell or non-human animal under varying conditions.

10. The method according to claim 22, wherein the fibrotic disease is polycystic ovarian syndrome.

11. The method according to claim 22, wherein the fibrotic disease is uterine leiomyomas.

12. The method according to claim 22, wherein the fibrotic disease is cardiac fibrosis.

13. The method according to claim 22, wherein the fibrotic disease is myocardial infarction-initiated.

14. A method for assessing collagen production during different stages of embryotic development, comprising:
a) constructing a transgenic nucleic acid construct, wherein said construct comprises nucleic acid sequences encoding:
i) a Colla1 gene-specific regulatory element, and wherein said regulatory element is inserted into the construct at a far upstream DNAse I-hypersensitive site;
ii) a Colla1 gene promoter; and
iii) a reporter;
b) transfecting the construct into a non-human animal capable of expressing the reporter during different stages of embryotic development; and
c) monitoring expression in said non-human animal during different stages of embryonic development.

15. A method for assessing differential collagen production in different tissues, comprising:
a) constructing a transgenic nucleic acid construct, wherein said construct comprises nucleic acid sequences encoding:
i) a Colla1 gene-specific regulatory element, and wherein said regulatory element is inserted into the construct at a far upstream DNAse I-hypersensitive site;
ii) a Colla1 gene promoter; and
iii) a reporter;
b) transfecting the construct into a non-human animal capable of differentially expressing the reporter in different tissues; and
c) monitoring expression of the reporter in different tissues in the non-human animal.

16. A transgenic non-human animal comprising the construct according to claim 1.

17. A cell comprising the construct according to claim 1.

18. A vector comprising the construct according to claim 1.
